(19) United States
(12) Patent Application Publication
(10) Pub. No.: US 2008/0241172 A1
(43) Pub. Date: Oct. 2, 2008

(54) Mutated Cholinesterase Sequences, Corresponding Nucleic Acids And Their Uses

(75) Inventors: Jean Massoulie, Le Vesinet (FR); Suzanne Bon born Frere, Paris (FR)

Correspondence Address: YOUNG & THOMPSON, 209 Madison Street, Suite 500, ALEXANDRIA, VA, 22314, US

(73) Assignee: CENTRE NATIONAL DE LA RECHERCHE SCIENTIFIQUE, PARIS CEDEX 16 (FR)

(21) Appl. No.: 11/662,921

(22) PCT Filed: Sep. 16, 2004
(86) PCT No.: PCT/EP2004/010988

§ 371 Date: May 15, 2007

(51) Int. Cl. A61K 39/00; C12N 9/16; C07K 14/00; C07H 21/04; A61P 43/00; C12N 15/63; C12N 5/06; A01K 67/027

(52) U.S. Cl. 424/184.1; 435/196; 530/324; 536/23.2; 435/320.1; 435/325; 800/14

(57) ABSTRACT

The present invention relates to the use of a peptide sequence to form oligomers, especially tetramers, of cholinesterases, said peptide sequence comprising: a peptide corresponding to SEQ ID NO: 4, wherein any one of amino acids of position 12 to position 19 of SEQ ID NO; 4 is replaced by a cysteine, any homologous sequence of said peptide, or any sequence derived from said peptide, or any fragment of one of the sequences defined above, on the condition that is possesses the property of forming oligomers of cholinesterases.

The present invention concerns new mutated cholinesterase sequences, and the corresponding nucleic acids encoding said new mutated sequences.

The present invention also concerns the uses of said sequences, in particular for the preparation of oligomers of cholinesterases.

In vertebrates, the acetylcholinesterase (AChE) gene generates several types of catalytic subunits through alternative splicing in the 3′ region of the transcripts (Sikorav et al., 1988; Li et al., 1991; Li et al., 1993). These subunits possess the same common catalytic domain, followed by distinct C-terminal peptides, r, h and t, characterising the AChE_{R}, AChE_{H} and AChE_{T} variants (Massoulié et al., 1993; Massouliéet al., 1998; Massouliéet al., 2002). In mammals, AChE_{R} subunits seem to be expressed mostly during embryogenesis and in the brain after stress (Legay et al., 1993; Kaufer et al., 1998); they correspond to a soluble, monomeric enzyme species. AChE_{H} subunits possess one or two cysteines and a GPI-addition signal in their C-terminal peptide: they generate GPI-anchored, disulfide-linked dimers, which represent a major fraction of AChE in Torpedo electric organs and muscles, and are expressed at the surface of blood cells in mammals (Bon et al., 1982; Futerman et al., 1985; Coussen et al., 1995; Coussen et al., 2001). AChE_{T} subunits are expressed in muscles and in the nervous system of higher vertebrates and therefore represent the functional cholinesterase species in the cholinergic system (Li et al., 1993; Legay et al., 1993; Krejci et al., 1999).

The C-terminal t peptide confers several characteristic properties to AChE_{T} subunits, allowing them to form a series of homo-oligomers (monomers, dimers, tetramers and higher oligomers) when expressed in transfected COS cells (Legay et al., 1993; Duval et al., 1992); some of these molecules are amphiphilic, i.e. interact with detergent micelles (Bon et al., 1988; Bon et al., 1991). AChE_{T} subunits also form hetero-oligomers with the collagen ColQ or with the transmembrane protein PRiMA (Krejci et al., 1997; Perrier et al., 2002); in mammals, these structural proteins anchor the major functional species of cholinesterases in the basal lamina of the neuromuscular junction and in neuronal cell membranes, respectively (Fernandez et al., 1996; Feng et al., 1999). In the collagen-tailed and hydrophobic-tailed forms, four catalytic AChE subunits are associated through their C-terminal t peptides with proline-rich domains (PRAD) localised in the N-terminal regions of COlQ or PRiMA (Perrier et al., 2002; Duval et al., 1992; Bon et al., 1997).

The t peptide of AChE consists of 40 residues, with a series of seven strictly conserved aromatic residues, including three evenly spaced tryptophans, as well as acidic and basic residues which are conserved or semi-conserved in most vertebrates (Massoulié et al., 1998). This peptide is necessary for the amphiphilic properties which characterize AChE_{T} subunits and some of their oligomers (T₁^{a}, T₂^{a}, T₄^{a}), for the formation of nonamphiphilic homotetramers (T₄^{na}) as well as for the heteromeric association of AChE_{T} subunits with Q_{N}, an N-terminal fragment of collagen COlQ which contains a proline-rich motif (PRAD), thus producing T₄-Q_{N} complexes (Bon et al., 1997; Bon et Massoulié, 1997).

The t peptide constitutes an autonomous interaction domain and was called the WAT (tryptophan (W), Amphiphilic Tetramerization) domain, because it can associate with a PRAD even in the absence of the catalytic domain; moreover, addition of at peptide at the C-terminus of foreign proteins, GFP (green fluorescent protein) and alkaline phosphatase, endowed them with amphiphilic properties and enabled them to form PRAD-associated tetramers (Simon et al., 1998). It is also known that the simultaneous presence of the t peptide and of mutations at the interface of AChE dimers, the “four helix bundle” (Sussman et al., 1991), prevents the secretion of AChE_{T} subunits (Morel et al., 2001). It was recently shown that the t peptide induces intracellular degradation through the ERAD (endoplasmic reticulum-associated degradation)/proteasome pathway, to different extents depending on the protein to which it is attached, and that aromatic residues are necessary for this effect (Belbeoc'h et al., 2003).

Recent spectroscopic studies showed that the t peptide is organized as an amphiphilic a helix, in which aromatic residues form a hydrophobic sector (Cottingham et al., 2003; Bon et al., 2004). In addition, an analysis of intercatenary disulfide bonds in the T₄-Q_{N} complex also demonstrated that the four t peptides are parallel and oriented in the same direction, opposite to that of the PRAD (Bon et al., 2004).

An aim of the present invention is to provide new peptide sequences of cholinesterases, capable of being used for the formation of oligomers of cholinesterases, said oligomers presenting a high lifetime in the body, after their injection in the blood circulation.

The present invention relates to the use of a peptide sequence to form oligomers, especially tetraxners, of cholinesterases, said peptide sequence comprising:
a peptide corresponding to SEQ ID NO: 4, wherein any one of amino acids of position 12 to position 19 of SEQ ID NO: 4 is replaced by a cysteine,
any homologous sequence of said peptide, preferably having an identity of at least approximately 50%, and preferably of at least 60%, with said peptide, and possessing the property of forming oligomers of cholinesterases,
or any sequence derived from said peptide, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of said peptide, with the proviso that said fragment contains the mutated cysteine.

The present invention relates to the use of a peptide sequence to form oligomers, especially tetramers, of cholinesterases, said peptide sequence comprising:
a peptide of SEQ ID NO: 2, corresponding to peptide SEQ ID NO: 4, wherein serine of position 19 is replaced by a cysteine,
any homologous sequence of SEQ ID NO: 2, preferably having an identity of at least approximately 50%, and preferably of at least 60%, with sequence SEQ ID NO: 2, and possessing the property of forming oligomers of cholinesterases,
or any sequence derived from SEQ ID NO: 2, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 2, with the proviso that said fragment contains the cysteine of position 19.

SEQ ID NO: 2 is a new mutated peptide corresponding to the t peptide of human AChE (SEQ ID NO: 4), wherein serine of position 19 is replaced by a cysteine.

Said mutation does not affect the catalytic activity of the corresponding cholinesterase.

Said mutation is preferably such that the secretory pathway of the cholinesterase is conserved.

According to an advantageous embodiment, the present invention relates to the use of a peptide sequence such as defined above, to form homo-oligomers of cholinesterases.

The tetramers according to the invention have a higher lifetime in the body, after their injection in the blood circulation, in particular with respect to monomers, dimers or trimers of cholinesterases such as described in the prior art. Furthermore, said stability can be measured according to methods described in the following publications: Kronman et al. (1995), Kronman et al. (2000), Chitlaru et al. (1998) and Chitlaru et al. (2002).

According to an advantageous embodiment, the present invention relates to the use of a peptide sequence such as defined above, wherein peptide of SEQ ID NO: 2 contains a cysteine in position 37.

The present invention relates to the use of a peptide sequence to form oligomers, especially tetramers, of cholinesterases, said peptide sequence comprising:
a peptide of SEQ ID NO: 23, corresponding to peptide SEQ ID NO: 4, wherein threonine of position 12 is replaced by a cysteine,
any homologous sequence of SEQ ID NO: 23, preferably having an identity of at least approximately 50%, and preferably of at least 60%, with sequence SEQ ID NO: 23, and possessing the property of forming oligomers of cholinesterases,
or any sequence derived from SEQ ID NO: 23, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 23, with the proviso that said fragment contains the cysteine of position 12.

SEQ ID NO: 23 is a new mutated peptide corresponding to the t peptide of human AChE (SEQ ID NO: 4), wherein threonine of position 12 is replaced by a cysteine.

The present invention relates to the use of a peptide sequence to form oligomers, especially tetramers, of cholinesterases, said peptide sequence comprising:
a peptide of SEQ ID NO: 24, corresponding to peptide SEQ ID NO: 4, wherein glutamate of position 13 is replaced by a cysteine,
any homologous sequence of SEQ ID NO: 24, preferably having an identity of at least approximately 50%, and preferably of at least 60%, with sequence SEQ ID NO: 24, and possessing the property of forming oligomers of cholinesterases,
or any sequence derived from SEQ ID NO: 24, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 24, with the proviso that said fragment contains the cysteine of position 13.

SEQ ID NO: 24 is a new mutated peptide corresponding to the t peptide of human AChE (SEQ ID NO: 4), wherein glutamate of position 13 is replaced by a cysteine.

The present invention relates to the use of a peptide sequence to form oligomers, especially tetramers, of cholinesterases, said peptide sequence comprising:
a peptide of SEQ ID NO: 25, corresponding to peptide SEQ ID NO: 4, wherein phenylalanine of position 14 is replaced by a cysteine,
any homologous sequence of SEQ ID NO: 25, preferably having an identity of at least approximately 50%, and preferably of at least 60%, with sequence SEQ ID NO: 25, and possessing the property of forming oligomers of cholinesterases,
or any sequence derived from SEQ ID NO: 25, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 25, with the proviso that said fragment contains the cysteine of position 14.

SEQ ID NO: 25 is a new mutated peptide corresponding to the t peptide of human AChE (SEQ ID NO: 4), wherein phenylalanine of position 14 is replaced by a cysteine.

The present invention relates to the use of a peptide sequence to form oligomers, especially tetramers, of cholinesterases, said peptide sequence comprising:
a peptide of SEQ ID NO: 26, corresponding to peptide SEQ ID NO: 4, wherein histidine of position 15 is replaced by a cysteine,
any homologous sequence of SEQ ID NO: 26, preferably having an identity of at least approximately 50%, and preferably of at least 60%, with sequence SEQ ID NO: 26, and possessing the property of forming oligomers of cholinesterases,
or any sequence derived from SEQ ID NO: 26, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 26, with the proviso that said fragment contains the cysteine of position 15.

SEQ ID NO: 26 is a new mutated peptide corresponding to the t peptide of human AChE (SEQ ID NO: 4), wherein histidine of position 15 is replaced by a cysteine.

The present invention relates to the use of a peptide sequence to form oligomers, especially tetramers, of cholinesterases, said peptide sequence comprising:
a peptide of SEQ ID NO: 27, corresponding to peptide SEQ ID NO: 4, wherein arginine of position 16 is replaced by a cysteine,
any homologous sequence of SEQ ID NO: 27, preferably having an identity of at least approximately 50%, and preferably of at least 60%, with sequence SEQ ID NO: 27, and possessing the property of forming oligomers of cholinesterases,
or any sequence derived from SEQ ID NO: 27, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 27, with the proviso that said fragment contains the cysteine of position 16.

SEQ ID NO: 27 is a new mutated peptide corresponding to the t peptide of human AChE (SEQ ID NO: 4), wherein arginine of position 16 is replaced by a cysteine.

The present invention relates to the use of a peptide sequence to form oligomers, especially tetramers, of cholinesterases, said peptide sequence comprising:
a peptide of SEQ ID NO: 28, corresponding to peptide SEQ ID NO: 4, wherein tryptophane of position 17 is replaced by a cysteine,
any homologous sequence of SEQ ID NO: 28, preferably having an identity of at least approximately 50%, and preferably of at least 60%, with sequence SEQ ID NO: 28, and possessing the property of forming oligomers of cholinesterases,
or any sequence derived from SEQ ID NO: 28, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 28, with the proviso that said fragment contains the cysteine of position 17.

SEQ ID NO: 28 is a new mutated peptide corresponding to the t peptide of human AChE (SEQ ID NO: 4), wherein tryptophane of position 17 is replaced by a cysteine.

The present invention relates to the use of a peptide sequence to form oligomers, especially tetramers, of cholinesterases, said peptide sequence comprising:
a peptide of SEQ ID NO: 29, corresponding to peptide SEQ ID NO: 4, wherein serine of position 18 is replaced by a cysteine,
any homologous sequence of SEQ ID NO: 29, preferably having an identity of at least approximately 50%, and preferably of at least 60%, with sequence SEQ ID NO: 29, and possessing the property of forming oligomers of cholinesterases,
or any sequence derived from SEQ ID NO: 29, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 29, with the proviso that said fragment contains the cysteine of position 18.

SEQ ID NO: 29 is a new mutated peptide corresponding to the t peptide of human AChE (SEQ ID NO: 4), wherein serine of position 18 is replaced by a cysteine.

The present invention relates to the use as defined above of said peptide sequence as a fusion protein with said cholinesterases to be oligomerized.

The present invention relates to the use as defined above, wherein the cholinesterases are chosen among those containing peptide of SEQ ID NO: 4 in their native amino acid sequence.

The present invention relates to the use as defined above, wherein the cholinesterases are chosen among those not containing peptide of SEQ ID NO: 4 in their native amino acid sequence.

The present invention relates to the use such as defined above, wherein said peptide sequence comprises also a linker of about 3 to about 10 amino acids, said linker being inserted upstream peptide of SEQ ID NO: 4.

The present invention relates to a protein, characterized in that it comprises or is constituted by:
a peptide corresponding to SEQ ID NO: 4, wherein any one of amino acids of position 12 to position 19 of SEQ ID NO: 4 is replaced by a cysteine,
any homologous sequence of said peptide, preferably having an identity of at least approximately 50%, and preferably of at least 60%, with said peptide, and possessing the property of forming oligomers of cholinesterases,
or any sequence derived from said peptide, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of said peptide, with the proviso that said fragment contains the mutated cysteine.

The present invention relates to a protein, characterized in that it comprises or is constituted by:
a peptide of SEQ ID NO:2,
any homologous sequence of SEQ ID NO: 2, preferably having an identity of at least approximately 50%, and preferably of at least 60%, with sequence SEQ ID NO: 2, and possessing the property of forming oligomers of cholinesterases,
or any sequence derived from SEQ ID NO: 2, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 2, with the proviso that said fragment contains the cysteine of position 19.

According to an advantageous embodiment, the present invention also relates to any homologous sequence, having an identity of at least approximately 56%, and preferably of at least 60%, and more preferably of at least 65%, and most preferably of at least 70% with sequence SEQ ID NO: 2, and possessing the property of forming oligomers of cholinesterases.

According to an advantageous embodiment, the present invention relates to a peptide sequence such as defined above, wherein peptide of SEQ ID NO: 2 contains a cysteine in position 37.

The present invention relates to a protein, characterized in that it comprises or is constituted by:
a peptide of SEQ ID NO: 23,
any homologous sequence of SEQ ID NO: 23, preferably having an identity of at least approximately 50%, and preferably of at least 60%, with sequence SEQ ID NO: 23, and possessing the property of forming oligomers of cholinesterases,
or any sequence derived from SEQ ID NO: 23, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 23, with the proviso that said fragment contains the cysteine of position 12.

The present invention relates to a protein, characterized in that it comprises or is constituted by:
a peptide of SEQ ID NO: 24,
any homologous sequence of SEQ ID NO: 24, preferably having an identity of at least approximately 50%, and preferably of at least 60%, with sequence SEQ ID NO: 24, and possessing the property of forming oligomers of cholinesterases,
or any sequence derived from SEQ ID NO: 24, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 24, with the proviso that said fragment contains the cysteine of position 13.

The present invention relates to a protein, characterized in that it comprises or is constituted by:
a peptide of SEQ ID NO: 25,
any homologous sequence of SEQ ID NO: 25, preferably having an identity of at least approximately 50%, and preferably of at least 60%, with sequence SEQ ID NO: 25, and possessing the property of forming oligomers of cholinesterases, or any sequence derived from SEQ ID NO: 25, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 25, with the proviso that said fragment contains the cysteine of position 14.

The present invention relates to a protein, characterized in that it comprises or is constituted by:
a peptide of SEQ ID NO: 26,
any homologous sequence of SEQ ID NO: 26, preferably having an identity of at least approximately 50%, and preferably of at least 60%, with sequence SEQ ID NO: 26, and possessing the property of forming oligomers of cholinesterases,
or any sequence derived from SEQ ID NO: 26, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 26, with the proviso that said fragment contains the cysteine of position 15.

The present invention relates to a protein, characterized in that it comprises or is constituted by:
a peptide of SEQ ID NO: 27,
any homologous sequence of SEQ ID NO: 27, preferably having an identity of at least approximately 50%, and preferably of at least 60%, with sequence SEQ ID NO: 27, and possessing the property of forming oligomers of cholinesterases,
or any sequence derived from SEQ ID NO: 27, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 27, with the proviso that said fragment contains the cysteine of position 16.

The present invention relates to a protein, characterized in that it comprises or is constituted by:
a peptide of SEQ ID NO: 28,
any homologous sequence of SEQ ID NO: 28, preferably having an identity of at least approximately 50%, and preferably of at least 60%, with sequence SEQ ID NO: 28, and possessing the property of forming oligomers of cholinesterases,
or any sequence derived from SEQ ID NO: 28, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 28, with the proviso that said fragment contains the cysteine of position 17.

The present invention relates to a protein, characterized in that it comprises or is constituted by:
a peptide of SEQ ID NO: 29,
any homologous sequence of SEQ ID NO: 29, preferably having an identity of at least approximately 50%, and preferably of at least 60%, with sequence SEQ ID NO: 29, and possessing the property of forming oligomers of cholinesterases,
or any sequence derived from SEQ ID NO: 29, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 29, with the proviso that said fragment contains the cysteine of position 18.

The present invention relates to a protein, characterized in that it comprises or is constituted by:
a peptide of SEQ ID NO: 2,
any homologous sequence of SEQ ID NO: 2, preferably having an identity of at least approximately 50%, and preferably of at least 60%, with sequence SEQ ID NO: 2, and possessing the property of forming oligomers of cholinesterases,
or any sequence derived from SEQ ID NO: 2, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 2, with the proviso that said fragment contains the cysteine of position 19,

with the proviso that, when said protein comprises said peptide of SEQ ID NO: 2, or said homologous or derived sequence, or said fragment, the flanking regions of said peptide, or said homologous or derived sequence, or said fragment are fragments of a cholinesterase.

According to an advantageous embodiment, the present invention relates to a peptide sequence such as defined above, wherein peptide of SEQ ID NO: 2 contains a cysteine in position 37.

According to an advantageous embodiment, the present invention relates to a homologous sequence, or derived sequence such as defined above, wherein peptide of SEQ ID NO: 2 contains a cysteine in position 37.

The present invention relates to a protein as defined above, characterized in that it is a cholinesterase whose native amino acid sequence contains peptide of SEQ ID NO: 4 or any homologous sequence of SEQ ID NO: 4, preferably having an identity of at least approximately 50%, and preferably of at least 60%, with sequence SEQ ID NO: 4.

The present invention relates to a protein as defined above, characterized in that it is a cholinesterase whose native amino acid sequence does not contain peptide of SEQ ID NO: 4 or any homologous sequence of SEQ ID NO: 4, preferably having an identity of at least approximately 50%, and preferably of at least 60%, with sequence SEQ ID NO: 4.

The present invention relates to a protein as defined above, having the following sequence: SEQ ID NO: 6, SEQ ID NO: 8, SEQ ID NO:10, SEQ ID NO:12 or SEQ ID NO: 14.

SEQ ID NO: 6 is a new protein corresponding to the mutated human AChE, wherein serine of position 593 is replaced by a cysteine.

SEQ ID NO: 8 is a new protein corresponding to the mutated human BChE, wherein asparagine of position 580 is replaced by a cysteine.

SEQ ID NO: 10 is a new protein corresponding to the mutated rat AChE, wherein serine of position 593 is replaced by a cysteine.

SEQ ID NO: 12 is a new protein corresponding to the mutated torpedo AChE, wherein serine of position 578 is replaced by a cysteine.

SEQ ID NO:14 is a new protein corresponding to the mutated murine AChE, wherein serine of position 593 is replaced by a cysteine.

The present invention relates to a protein as defined above, characterized in that it is constituted by peptide of SEQ ID NO: 2.

Such a protein corresponds to the whole t peptide of human AChE.

The present invention relates to a protein as defined above, characterized in that it is constituted by peptide of the following sequence: SEQ ID NO: 2, SEQ ID NO: 16, SEQ ID NO: 18, SEQ ID NO: 20 or SEQ ID NO: 22.

SEQ ID NO: 2 is a new mutated peptide corresponding to mutated t peptide of human AChE, wherein serine of position 19 is replaced by a cysteine; this new mutated peptide is a fragment of SEQ ID NO: 6, delimited from the amino acid in position (575) to the amino acid in position (614) of the sequence SEQ ID NO: 6.

SEQ ID NO: 16 is a new mutated peptide corresponding to mutated t peptide of human BChE, wherein asparagine of position 19 is replaced by a cysteine; this new mutated peptide is a fragment of SEQ ID NO: 8, delimited from the amino acid in position (562) to the amino acid in position (602) of the sequence SEQ ID NO: 8.

SEQ ID NO: 18 is a new protein corresponding to mutated t peptide of rat AChE, wherein serine of position 19 is replaced by a cysteine; this new mutated peptide is a fragment of SEQ ID NO: 10, delimited from the amino acid in position (575) to the amino acid in position (614) of the sequence SEQ ID NO: 10.

SEQ ID NO: 20 is a new protein corresponding to mutated t peptide of torpedo AChE, wherein serine of position 19 is replaced by a cysteine; this new mutated peptide is a fragment of SEQ ID NO: 12, delimited from the amino acid in position (560) to the amino acid in position (599) of the sequence SEQ ID NO: 12.

SEQ ID NO: 22 is a new protein corresponding to mutated t peptide of murine AChE, wherein serine of position 19 is replaced by a cysteine; this new mutated peptide is a fragment of SEQ ID NO: 14, delimited from the amino acid in position (575) to the amino acid in position (614) of the sequence SEQ ID NO: 14.

The present invention also relates to a DNA sequence coding for a protein as defined above.

The present invention also relates a DNA sequence, which comprises or is constituted by:
nucleotide sequence SEQ ID NO: 1,
or any nucleotide sequence derived, by degeneration of the genetic code, from the sequence SEQ ID NO: 1 coding for a protein represented by SEQ ID NO: 2,
or any nucleotide sequence derived, in particular by substitution, suppression or addition of one or more nucleotides, from the sequence SEQ ID NO:1 coding for a protein derived from SEQ ID NO: 2, as defined above,
or any homologous nucleotide sequence of SEQ ID NO:1, preferably having an identity of at least approximately 50%, and preferably of about 60%, with the sequence SEQ ID NO:1 coding for a homologous protein of SEQ ID NO: 2, as defined above,
or any fragment of the nucleotide sequence SEQ ID NO:1 or of the nucleotide sequences defined above, said fragment being preferably constituted of at least approximately 60 nucleotides adjacent in said sequence,
or any complementary nucleotide sequence of the abovementioned sequences or fragments,
or any nucleotide sequence capable of hybridizing in stringent conditions with the complementary sequence of one of the abovementioned sequences or fragments.

SEQ ID NO: 1 is a new nucleotide sequence coding for mutated protein SEQ ID NO: 2.

The present invention also relates to a DNA sequence such as defined above, characterized in that it has the following sequence: SEQ ID NO: 5, SEQ ID NO: 7, SEQ ID NO: 9, SEQ ID NO:11 or SEQ ID NO:13.

SEQ ID NO: 5 is a new nucleotide sequence coding for mutated protein SEQ ID NO: 6.

SEQ ID NO: 7 is a new nucleotide sequence coding for mutated protein SEQ ID NO: 8.

SEQ ID NO: 9 is a new nucleotide sequence coding for mutated protein SEQ ID NO:10.

SEQ ID NO:11 is a new nucleotide sequence coding for mutated protein SEQ ID NO:12.

SEQ ID NO:13 is a new nucleotide sequence coding for mutated protein SEQ ID NO:14.

The present invention also relates to a recombinant vector, in particular plasmid, cosmid, phage or virus DNA, containing a DNA sequence such as defined above.

The present invention also relates to a recombinant vector such as defined above, containing the elements necessary for the expression in a host cell of polypeptides coded by the nucleic acids such as defined previously, inserted into said vector.

The present invention also relates to a host cell, chosen in particular from bacteria, viruses, yeasts, fungi, plants, vertebrate cells or mammalian cells, said host cell being transformed using a recombinant vector such as defined above.

The present invention also relates to a non-human mammalian transgenic animal, especially a goat, comprising non-human mammalian cells which have been transformed using a recombinant vector such as defined above.

The present invention also relates to a pharmaceutical or vaccine composition, characterized in that it comprises a protein such as defined previously, or a DNA sequence such as defined previously, in association with a pharmaceutically acceptable vehicle.

According to an advantageous embodiment, the present invention relates to a pharmaceutical composition comprising from 100 to 300 mg, and preferably from 200 to 250 mg of the protein such as defined previously pro 70 kg body weight (Ashani and Pistinner, 2004).

The present invention also relates to the use of a protein such as defined previously, to prevent the toxic effects of organophosphate poisons.

Said organophosphate poisons in particular belong to the group comprising sarin, tabun, soman, 7-(methylethoxyphosphinyloxy)-1-methyl-quinolinium iodide (MEPQ) or VX (Doctor et al., 1991; Maxwell et al., 1992; Raveh et al., 1989).

### FIGURES

FIG. 1 shows the oligomeric forms obtained with cysteines at different positions in the t peptide and the corresponding sedimentation patterns of cellular and secreted molecular forms in gradients containing Triton X-100 (−) and Brij-97 (− −). The areas under the profiles are proportional to the corresponding activities.

### MATERIALS AND METHODS

AChE Constructs and Site Directed Mutagenesis

Mutagenesis was performed by the method of Kunkel and coll. (Kunkel et al., 1987). cDNAs encoding wild type and mutated Torpedo AChE_{T}, intact or deleted of its PRAD motif (residues 70-86), were inserted in the pEFBos vector. The residues of the t peptide are numbered from 1 to 40, so that the Torpedo mutants are indicated by the modified residues, e.g. W17P.

Mutagenesis

Single stranded DNA was prepared by expressing the plasmid vector in RZ 1032 bacteria together with helper phage (M13KO7), using standard procedures. The single stranded DNA was hybridized with phosphorylated mutagenic oligonucleotide (20- to 30-mer), and the complementary strand was synthesized by T7 polymerase, circularized with T4 ligase, and used to transform DH5a competent E. coli bacteria to produce mutated vectors. Several clones were selected and screened for the mutation, which was associated with the change of a restriction site. The entire coding sequences of selected clones were verified. Mutated vectors were expressed in transfected COS cells and the oligomeric state of the resulting enzyme was determined by sedimentation in sucrose gradients (Belbeoc'h et al., 2004).

Transfection of COS Cells

COS cells were transfected by the DEAE-dextran method, as previously described (Bon et al., 1997), using 4 μg of DNA encoding the AChE catalytic subunit and 4 μg of DNA encoding Q_{N} or PRAD-deleted Q_{N}, per 100 mm dish. Because Torpedo AChE folds into its active conformation at 27° C., but not at 37° C., the cells were incubated for two days at 37° C. after transfection, then transferred to 27° C. and maintained at this temperature for 3-4 days, in a medium containing 10% Nuserum (Inotech, Dottikon, Switzerland), which had been pretreated with 10⁻⁶ M soman to inactivate serum choline sterases.

To analyze its heteromeric interaction with an associated structural protein, AChE_{T} was co-expressed with Q_{N}. By using Q_{N} rather than full length ColQ, we avoid the complexity due to the formation of the triple helical collagen and to the low salt aggregation of collagen-tailed AChE forms (Bon et al., 2003). A flag epitope (DYKDDDDK) was added at the C-terminus of Q_{N}, so that complexes containing this protein could be characterized with the anti-flag antibody M2 (Kodak), as previously described (Bon et al., 1997). The effect of Q_{N} on the level of cellular and secreted activity was analysed by comparing co-expression of AChE_{T} with full length Q_{N} and with a PRAD-deleted Q_{N}, to compensate for competition between the two transfected vectors.

Cell Extracts

The cells were extracted at 20° C. with TMg buffer (1% Triton X-100, 50 mM Tris-HCl, pH 7.5, 10 mM MgCl₂), and then centrifuged at 13,000 rpm for 30 min. Media were also centrifuged at 13,000 rpm for 30 min to remove cell debris before analysis.

Enzyme Assays

AChE activity was determined by the colorimetric method of Ellman et al. (Ellman et al., 1961) at room temperature; because the monomeric Torpedo AChE forms produced by some mutants were inactivated by DTNB (Morel et al., 1999), the enzyme samples were incubated for variable periods of time, depending on their activity, with a reaction medium containing acetylthiocholine iodide in phosphate buffer, pH 7; DTNB was then added and the optical density at 414 nm was determined with a Labsystems Multiskan RC automatic plate reader (Helsinki, Finland). Alkaline phosphatase and β-galactosidase from E. coli were assayed with the chromogenic substrates p-nitrophenyl phosphate and o-nitrophenyl galactoside, respectively.

Sedimentation and Electrophoretic Analyses

Centrifugation was performed in 5-20% sucrose gradients (50 mM Tris-HCl, pH 7.5, 50 mM MgCl₂, either in the presence of 0.2% Brij-97 or in the presence of 0.2% Triton X-100) in a Beckman SW41 rotor, at 36,000 rpm, for 18 h at 6° C. The gradients contained Escherichia coli β-galactosidase (16 S) and alkaline phosphatase (6.1 S) as internal sedimentation standards (Bon et al., 1997). Amphiphilic molecules generally sediment faster in the presence of Triton X100 than of Brij-97, providing an indication on their amphiphilic character.

Electrophoresis in non-denaturating polyacrylamide gels were performed as described by Bon et al. (1988), and AChE activity was revealed by the histochemical method of Karnovsky and Roots (1964). In charge shift electrophoresis, the electrophoretic migration of amphiphilic molecules was accelerated in the presence of Na⁺ deoxycholate, when compared to migration in the presence of the neutral detergent Triton X-100 alone. As an index of the degree of amphiphilicity, the ratio between migration in the presence of DOC to migration in Triton X100 alone was used, after normalizing these migrations to that of a nonamphiphilic species, the wild type tetramers T₄^{na} or T₄-Q_{N}.

Both sedimentation and non-denaturing electrophoresis provide semi-quantitative information on the interaction of AChE molecules with micelles, and are generally in complete agreement.

Results

Analyses of Ache Activity and Molecular Forms

The inventors analyzed how mutations in the t peptide affect the levels of cellular and secreted activity of Torpedo AChE in transfected COS cells. The activities were normalized to those obtained for wild type AChE_{T} in parallel transfections. Immunofluorescence of the protein produced at early stages after transfection indicated that all mutants were expressed in a similar manner. After two days at 27° C., a temperature which allows a correct folding of active Torpedo AChE (see Methods), the level of cellular activity reached a plateau and the rate of secretion remained constant.

Effect of a Cysteine at Various Positions in the t Peptide

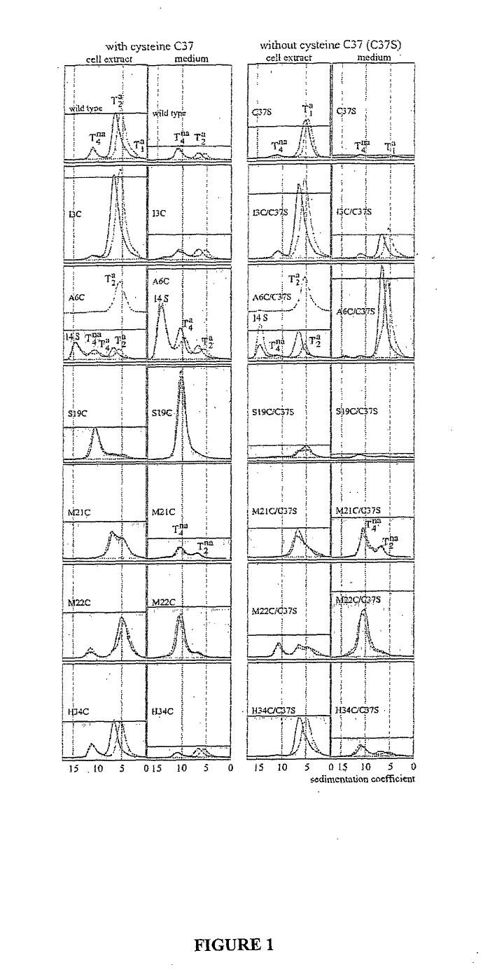

The formation of intercatenary disulfide bonds between wild type AChE_{T} subunits depends on the free cysteine residue located near the C-terminus of the t peptide, C37. Mutation of this cysteine to a serine reduced both cellular and secreted activities; it suppressed the formation of dimers and reduced cellular and secreted tetramers (FIG. 1); in the presence of Q_{N}, the secretion of T₄-Q_{N} complexes was reduced to about 75% of the wild type. Thus, the presence of an intercatenary disulfide bond appears necessary for dimerisation, but not for tetramerisation, particularly in the presence of Q_{N}.

To determine whether cysteines at other positions could allow dimerisation and further oligomerisation, we replaced residues 13, A6, T12, S19, M21, M22 or H34 by a cysteine, with or without mutation of C37 (C37S)(FIG. 1). Unlike C37S, none of these mutants produced monomers without dimers; therefore, when two cysteines were present, they were not engaged in an intracatenary disulfide bond but could form intercatenary bonds in dimers.

A cysteine at position 19, in the aromatic-rich segment but opposite to the aromatic cluster, had very different effects depending on the presence of cysteine C37. Without C37, mutant S19C/C37S produced lower levels of cellular or secreted activity. In contrast, mutant S19C (containing two cysteines at positions 19 and 37) showed a high level of secretion, mostly as nonamphiphilic tetramers, as observed for T12C.

### REFERENCES

Ashani, Y. & Pistinner, S. (2004) Estimation of the upper limit of human butyrylcholinesterase dose required for protection against organophosphates toxicity: a mathematically based toxicokinetic model. Toxicology Sciences. 77, 358-367;
Belbeoc'h, S., Massoulié, J. & Bon, S. (2003) The C-terminal T peptide of acetylcholinesterase enhances degradation of unassembled active subunits through the ERAD pathway. EMBO J. 22, 3536-3545;
Belbeoc'h, S., Falasca, C., Leroy, J., Ayon, A., Massoulié, J. & Bon, S. (2004) Elements of the C-terminal t peptide of acetylcholinesterase that determine amphiphilicity, homomeric and heteromeric associations, secretion and degradation. Eur. J. Biochem. 271, 1476-1487;
Bon, S. & Massoulié, J. (1997) Quaternary associations of acetylcholinesterase. I Oligomeric associations of T subunits with and without the amino-terminal domain of the collagen tail. J. Biol. Chem. 272, 3007-3015;
Bon, S. (1982) Molecular forms of acetylcholinesterase in developing Torpedo embryos. Neurochem. Int. 4, 577-585;
Bon, S., Coussen, F. & Massoulié, J. (1997) Quaternary associations of acetylcholinesterase; II. the polyproline attachment domain of the collagen tail. J. Biol. Chem. 272, 3016-3021;
Bon, S., Ayon, A., Leroy, J. & Massoulié, J. (2003) Trimerization domain of the collagen tail of acetylcholinesterase. Neurochem. Res. 28, 523-535;
Bon, S., Dufourcq, J., Leroy, J., Comut, I. & Massoulié, J. (2004) The C-terminal t peptide of acetylcholinesterase forms an alpha helix that supports homomeric and heteromeric interactions. Eur. J. Biochem. 271, 33-47;
Bon, S., Rosenberry, T. L. & Massoulié, J. (1991) Amphiphilic, glycophosphatidylinositol-specific phospholipase C (PI-PLC)-insensitive monomers and dimers of acetylcholinesterase. Cell. Mol. Neurobiol. 11, 157-172;
Bon, S., Toutant, J. P., Meflah, K. & Massoulié, J. (1988) Amphiphilic and nonamphiphilic forms of Torpedo cholinesterases: II. Electrophoretic variants and phosphatidylinositol phospholipase C— sensitive and -insensitive forms. J. Neurochem. 51, 786-794;
Chitlaru, T., Kronman, C., Zeevi, M., Kam, M., Harel, A., Ordentlich, A., Velan, B. & Shafferman, A. (1998) Modulation of circulatory residence of recombinant acetylcholinesterase through biochemical or genetic manipulation of sialylation levels. Biochem. J. 336, 647-658;
Chitlaru, T., Kronman, C., Velan, B. & Shafferman, A. (2002) Overloading and removal of N-glycosylation targets on human acetylcholinesterase: effects on glycan composition and circulatory residence time. Biochem. J. 363, 619-631;
Cottingham, M. G., Voskuil, J. L. A. & Vaux, D. J. T. (2003) The intact human acetylcholinesterase C-terminal oligomerization domain is alpha-helical in situ and in isolation, but a shorter fragment forms beta-sheet-rich amyloid fibrils and protofibrillar oligomers. Biochemistry. 42, 10863-10873;
Coussen, F., Ayon, A., Le Goff, A., Leroy, J., Massoulié, J. & Bon, S. (2001) Addition of a glycophosphatidylinositol to acetylcholinesterase; processing, degradation, and secretion. J. Biol. Chem. 276, 27881-27892;
Coussen, F., Bonnerot, C. & Massoulié, J. (1995) Stable expression of acetylcholinesterase and associated collagenic subunits in transfected RBL cell lines: production of GPI-anchored dimers and collagen-tailed forms. Eur. J. Cell Biol. 67, 254-260;
Doctor, B. P., Raveh, L., Wolfe, A. D., Maxwell, D. M. & Ashani, Y. (1991) Enzymes as pretreatment drugs for organophosphate toxicity. Neuroscience & Biobehavioral Reviews. 15, 123-128;
Duval, N., Krejci, E., Grassi, J., Coussen, F., Massoulié, J. & Bon, S. (1992) Molecular architecture of acetylcholinesterase collagen-tailed forms; construction of a glycolipid-tailed tetramer. EMBO J. 11, 3255-3261;
Duval, N., Massoulié, J. & Bon, S. (1992) H and T subunits of acetylcholinesterase from Torpedo, expressed in COS cells, generate all types of globular forms. J. Cell Biol. 118, 641-653;
Ellman, G. L., Courtney, K. D., Andres, V. & Featherstone, R. M. (1961) A new and rapid colorimetric determination of acetylcholinesterase activity. Biochem. Pharmacol. 7, 88-95;
Feng, G., Krejci, E., Molgo, J., Cunningham, J. M., Massoulié, J. & Sanes, J. R. (1999) Genetic analysis of collagen Q: Roles in acetylcholinesterase and butyrylcholinesterase assembly and in synaptic structure and function. J. Cell Biol. 144, 1349-1360;
Fernandez, H. L., Moreno, R. D. & Inestrosa, N. C. (1996) Tetrameric (G4) acetylcholinesterase: structure, localization, and physiological regulation. J. Neurochem. 66, 1335-1346;
Futerman, A. H., Low, M. G., Michaelson, D. M. & Silman, I. (1985) Solubilization of membrane-bound acetylcholinesterase by a phosphatidylinositol-specific phospholipase C. J. Neurochem. 45, 1487-1494;
Karnovsky, M. J. & Roots, L. (1964) A direct-coloring thiocholine method for cholinesterase. J. Histochem. Cytochem. 12, 219-222;
Kaufer, D., Friedman, A., Seidman, S. & Soreq, H. (1998) Acute stress facilitates long-lasting changes in cholinergic gene expression. Nature. 393, 373-377;
Krejci, E., Legay, C., Thomine, S., Sketelj, J. & Massoulié, J. (1999) Differences in expression of acetylcholinesterase and collagen Q control the distribution and oligomerization of the collagen-tailed forms in fast and slow muscles. J. Neurosci. 19, 10672-10679;
Krejci, E., Thomine, S., Boschetti, N., Legay, C., Sketelj, J. & Massoulié, J. (1997) The mammalian gene of acetylcholinesterase-associated collagen. J. Biol. Chem. 272, 22840-22847;
Kronman, C., Velan, B., Marcus, D., Ordentlich, A., Reuveny, S. & Shafferman, A. (1995) Involvement of oligomerization, N-glycosylation and sialylation in the clearance of cholinesterases from the circulation. Biochem. J. 311, 959-967;
Kronman, C., Chitlaru, T., Elhanany, E., Velan, B. & Shafferman, A. (2000) Hierarchy of post-translational modifications involved in the circulatory longevity of glycoproteins. J. Biol. Chem. 275, 29488-29502;
Kunkel, T. A., Roberts, J. D. & Zakour, R. A. (1987) Rapid and efficient site-specific mutagenesis without phenotypic selection. Methods Enzymol. 154, 367-382;
Legay, C., Bon, S. & Massoulié, J. (1993) Expression of a cDNA encoding the glycolipid-anchored form of rat acetylcholinesterase. FEBS Lett. 315, 163-166;
Legay, C., Bon, S., Vernier, P., Coussen, F. & Massoulié, J. (1993) Cloning and expression of a rat acetylcholinesterase subunit: generation of multiple molecular forms and complementarity with a Torpedo collagenic subunit. J. Neurochem. 60, 337-346;
Li, Y., Camp, S. & Taylor, P. (1993) Tissue-specific expression and alternative mRNA processing of the mammalian acetylcholinesterase gene. J. Biol. Chem. 268, 5790-5797;
Li, Y., Camp, S., Rachinsky, T. L., Getman, D. & Taylor, P. (1991) Gene structure of mammalian acetylcholinesterase. Alternative exons dictate tissue-specific expression. J. Biol. Chem. 266, 23083-23090;
Massoulié, J. (2002) The origin of the molecular diversity and functional anchoring of cholinesterases. NeuroSignals. 11, 130-143;
Massoulié, J., Anselmet, A., Bon, S., Krejci, E., Legay, C., Morel, N. & Simon, S. (1998) Acetylcholinesterase: C-terminal domains, molecular forms and functional localization. J. Physiol. (Paris). 92, 183-190;
Massoulié, J., Pezzementi, L., Bon, S., Krejci, E. & Vallette, F. M. (1993) Molecular and cellular biology of cholinesterases. Prog. Neurobiol. 41, 31-91;
Maxwell, D. M., Castro, C. A., De La Hoz, D. M., Gentry, M. K., Gold, M. B., Solana, R. P., Wolfe, A. D. & Doctor, B. P. (1992) Protection of rhesus monkeys against soman and prevention of performance decrement by pretreatment with acetylcholinesterase. Toxicology and Applied Pharmacology. 115, 44-49;
Morel, N., Bon, S., Greenblatt, H. M., Van Belle, D., Wodak, S. J., Sussman, J. L., Massoulié, J. & Silman, I. (1999) Effect of mutations within the peripheral anionic site on the stability of acetylcholinesterase. Mol. Pharmacol. 55, 982-992;
Morel, N., Leroy, J., Ayon, A., Massoulié, J. & Bon, S. (2001) Acetylcholinesterase H and T dimers are associated through the same contact; mutations at this interface interfere with the C-terminal T peptide, inducing degradation rather than secretion. J. Biol. Chem. 276, 37379-37389;
Perrier, A. L., Massoulié, J. & Krejci, E. (2002) PRiMA, the membrane anchor of acetylcholinesterase in brain. Neuron. 33, 275-285;
Raveh, L., Ashani, Y., Levy, D., De La Hoz, D., Wolfe, A. D. & Doctor, B. P. (1989) Acetylcholinesterase prophylaxis against organophosphate poisoning.—quantitative correlation between protection and blood-enzyme level in mice. Biochemical Pharmacology. 38(3), 529-534;
Sikorav, J. L., Duval, N., Anselmet, A., Bon, S., Krejci, E., Legay, C., Osterlund, M., Reimund, B. & Massoulié, J. (1988) Complex alternative splicing of acetylcholinesterase transcripts in Torpedo electric organ; primary structure of the precursor of the glycolipid-anchored dimeric form. EMBO J. 7, 2983-2993;
Simon, S., Krejci, E. & Massoulié, J. (1998) A four-to-one association between peptide motifs: four C-terminal domains from cholinesterase assemble with one proline-rich attachment domain (PRAD) in the secretory pathway. EMBO J. 17, 6178-6187;
Sussman, J. L., Harel, M., Frolow, F., Oefner, C., Goldman, A., Toker, L. & Silman, I. (1991) Atomic structure of acetylcholinesterase from Torpedo californica: a prototypic acetylcholine-binding protein. Science. 253, 872-879.

1-23. (canceled)

24. A process for the preparation of, especially tetramers, of cholinesterases, comprising the use of a peptide sequence comprising:
a peptide corresponding to SEQ ID NO: 4, wherein any one of amino acids of position 12 to position 19 of SEQ ID NO: 4 is replaced by a cysteine,
any homologous sequence of said peptide, preferably having an identity of at least approximately 50%, and preferably of at least 60%, with said peptide, and possessing the property of forming oligomers of cholinesterases,
or any sequence derived from said peptide, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of said peptide, with the proviso that said fragment contains the mutated cysteine.

25. The process of claim 24, wherein the peptide sequence comprises:
a peptide of SEQ ID NO: 2, corresponding to peptide
SEQ ID NO: 4, wherein serine of position 19 is replaced by a cysteine,
any homologous sequence of SEQ ID NO: 2, preferably having an identity of at least approximately 50%, and preferably of at least 60%, with sequence SEQ ID NO: 2, and possessing the property of forming oligomers of cholinesterases,
or any sequence derived from SEQ ID NO: 2, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of SEQ ID NO: 2, with the proviso that said fragment contains the cysteine of position 19.

26. The process of claim 24 comprising the use of a peptide sequence as a fusion protein with said cholinesterases to be oligomerized.

27. The process of claim 24, wherein the cholinesterases are chosen among those containing peptide of SEQ ID NO: 4 in their native amino acid sequence.

28. The process of claim 1, wherein the cholinesterases are chosen among those not containing peptide of SEQ ID NO: 4 in their native amino acid sequence.

29. The process of claim 24, wherein said peptide sequence comprises also a linker of about 3 to about 10 amino acids, said linker being inserted upstream peptide of SEQ ID NO: 4.

30. A protein, characterized in that said protein comprises or is constituted by:
a peptide corresponding to SEQ ID NO: 4, wherein any one of amino acids of position 12 to position 19 of SEQ ID NO: 4 is replaced by a cysteine,
any homologous sequence of said peptide, preferably having an identity of at least approximately 50%, and preferably of at least 60%, with said peptide, and possessing the property of forming oligomers of cholinesterases,
or any sequence derived from said peptide, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence of said peptide, with the proviso that said fragment contains the mutated cysteine.

31. A protein, characterized in that said protein comprises or is constituted by:
a peptide of SEQ ID NO: 2,
any homologous sequence of SEQ ID NO: 2, preferably having an identity of at least approximately 50%, and preferably of at least 60%, with sequence SEQ ID NO: 2, and possessing the property of forming oligomers of cholinesterases,
or any sequence derived from SEQ ID NO: 2, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 2, with the proviso that said fragment contains the cysteine of position 19.

32. A protein, characterized in that said protein comprises or is constituted by:
a peptide of SEQ ID NO: 2,
any homologous sequence of SEQ ID NO: 2, preferably having an identity of at least approximately 50%, and preferably of at least 60%, with sequence SEQ ID NO: 2, and possessing the property of forming oligomers of cholinesterases,
or any sequence derived from SEQ ID NO: 2, by substitution, suppression or addition of one or more amino acids, having the property of forming oligomers of cholinesterases,
or any fragment of one of the sequences defined above, on the condition that it possesses the property of forming oligomers of cholinesterases, in particular any fragment being constituted by at least approximately 20 contiguous amino acids in the sequence SEQ ID NO: 2, with the proviso that said fragment contains the cysteine of position 19,
with the proviso that, when said protein comprises said peptide of SEQ ID NO: 2, or said homologous or derived sequence, or said fragment, the flanking regions of said peptide, or said homologous or derived sequence, or said fragment are fragments of a cholinesterase.

33. A protein according to claim 30, characterized in that said protein is a cholinesterase whose native amino acid sequence contains peptide of SEQ ID NO: 4 or any homologous sequence of SEQ ID NO: 4, preferably having an identity of at least approximately 50%, and preferably of at least 60%, with sequence SEQ ID NO: 4.

34. A protein according to claim 30, characterized in that it is a cholinesterase whose native amino acid sequence does not contain peptide of SEQ ID NO: 4 or any homologous sequence of SEQ ID NO: 4, preferably having an identity of at least approximately 50%, and preferably of at least 60%, with sequence SEQ ID NO: 4.

35. A protein of claim 32, having the following sequence: SEQ ID NO: 6, SEQ ID NO: 8, SEQ ID NO:10, SEQ ID NO:12 or SEQ ID NO:14.

36. A protein of claim 32, characterized in that it is constituted by peptide of SEQ ID NO: 2.

37. A protein of claim 32, characterized in that
said peptide is constituted by peptide of the
following sequence: SEQ ID NO: 2, SEQ ID NO:16,
SEQ ID NO:18, SEQ ID NO: 20 or SEQ ID NO: 22.

38. A DNA sequence coding for a protein according to claim 30.

39. A DNA sequence which comprises or is constituted by:
nucleotide sequence SEQ ID NO:1,
or any nucleotide sequence derived, by degeneration of the genetic code, from the sequence SEQ ID NO:1 coding for a protein represented by SEQ ID NO: 2,
or any nucleotide sequence derived, in particular by substitution, suppression or addition of one or more nucleotides, from the sequence SEQ ID NO:1 coding for a protein derived from SEQ ID NO: 2,
or any homologous nucleotide sequence of SEQ ID NO:1, preferably having an identity of at least approximately 60% with the sequence SEQ ID NO:1 coding for a homologous protein of SEQ ID NO: 2,
or any fragment of the nucleotide sequence SEQ ID NO:1 or of the nucleotide sequences defined above, said fragment being preferably constituted of at least approximately 60 nucleotides adjacent in said sequence,
or any complementary nucleotide sequence of the abovementioned sequences or fragments,
or any nucleotide sequence capable of hybridizing in stringent conditions with the complementary sequence of one of the abovementioned sequences or fragments.

40. A DNA sequence according to claim 39, characterized in that said peptide has the following sequence: SEQ ID NO: 5, SEQ ID NO: 7, SEQ ID NO: 9, SEQ ID NO:11 or SEQ ID NO:13.

41. A recombinant vector, in particular plasmid, cosmid, phage or virus DNA, containing a DNA sequence according to claim 38.

42. A recombinant vector, containing the elements necessary for the expression in a host cell of polypeptides coded by the nucleic acids according to claim 38, inserted into said vector.

43. A host cell, chosen in particular from bacteria, viruses, yeasts, fungi, plants, vertebrate cells or mammalian cells, said host cell being transformed using a recombinant vector according to claim 41.

44. A non-human mammalian transgenic animal, especially a goat, comprising non-human mammalian cells which have been transformed using a recombinant vector according to claim 41.

45. A pharmaceutical or vaccine composition, characterized in that it comprises a protein according to claim 30, or a DNA sequence encoding for said peptide, in association with a pharmaceutically acceptable vehicle.

46. A process for preventing the toxic effects of organophosphate poisons, comprising the use of a protein according to claim 30.


SEQUENCE LISTING (30 sequences)

SEQ ID NO: 1 - DNA, 123 bp, Homo sapiens
  CDS (1)..(120)
gacacgctcgacgaggcggagcgccagtggaaggccgagttccaccgctggagctgytacatggtgcactggaagaaccagttcgaccactacagcaagcaggatcgctgctcagacctgtga

SEQ ID NO: 2 - protein, 40 aa, Homo sapiens
DTLDEAERQWKAEFHRWSCYMVHWKNQFDHYSKQDRCSDL

SEQ ID NO: 3 - DNA, 123 bp, Homo sapiens
  CDS (1)..(120)
gacacgctcgacgaggcggagcgccagtggaaggccgagttccaccgctggagctcctacatggtgcactggaagaaccagttcgaccactacagcaagcaggatcgctgctcagacctgtga

SEQ ID NO: 4 - protein, 40 aa, Homo sapiens
DTLDEAERQWKAEFHRWSSYMVHWKNQFDHYSKQDRCSDL

SEQ ID NO: 5 - DNA, 2218 bp, Homo sapiens
  CDS (157)..(1998)
ctctcccctcatctttgccaacctgccccacctcctctgcagctgagcgataacccttgggccgacagtgccctaatctcctccctcctggcttctcgaccgacccttcaccctttccctttctttctcccagcagacgccgcctgccctgcagccatgaggcccccgcagtgtctgctgcacacgccttccctggcttccccactccttctcctcctcctctggctcctgggtggaggagtgggggctgagggccgggaggatgcagagctgctggtgacggtgcgtgggggccggctgcggggcattcgcctgaagacccccgggggccctgtctctgctttcctgggcatcccctttgcggagccacccatgggaccccgtcgctttctgccaccggagcccaagcagccttggtcaggggtggtagacgctacaaccttccagagtgtctgctaccaatatgtggacaccctatacccaggttttgagggcaccgagatgtggaaccccaaccgtgagctgagcgaggactgcctgtacctcaacgtgtggacaccatacccccggcctacatcccccacccctgtcctcgtctggatctatgggggtggcttctacagtggggcctcctccttggacgtgtacgatggccgcttcttggtacaggccgagaggactgtgctggtgtccatgaactaccgggtgggagcctttggcttcctggccctgccggggagccgagaggccccgggcaatgtgggtctcctggatcagaggctggccctgcagtgggtgcaggagaacgtggcagccttcgggggtgacccgacatcagtgacgctgtttggggagagcgcgggagccgcctcggtgggcatgcacctgctgtccccgcccagccggggcctgttccacagggccgtgctgcagagcggtgcccccaatggaccctgggccacggtgggcatgggagaggcccgtcgcagggccacgcagctggcccaccttgtgggctgtcctccaggcggcactggtgggaatgacacagagctggtagcctgccttcggacacgaccagcgcaggtcctggtgaaccacgaatggcacgtgctgcctcaagaaagcgtcttccggttctccttcgtgcctgtggtagatggagacttcctcagtgacaccccagaggccctcatcaacgcgggagacttccacggcctgcaggtgctggtgggtgtggtgaaggatgagggctcgtattttctggtttacggggccccaggcttcagcaaagacaacgagtctctcatcagccgggccgagttcctggccggggtgcgggtcggggttccccaggtaagtgacctggcagccgaggctgtggtcctgcattacacagactggctgcatcccgaggacccggcacgcctgagggaggccctgagcgatgtggtgggcgaccacaatgtcgtgtgccccgtggcccagctggctgggcgactggctgcccagggtgcccgggtctacgcctacgtctttgaacaccgtgcttccacgctctcctggcccctgtggatgggggtgccccacggctacgagatcgagttcatctttgggatccccctggacccctctcgaaactacacggcagaggagaaaatcttcgcccagcgactgatgcgatactgggccaactttgcccgcacaggggatcccaatgagccccgagaccccaaggccccacaatggcccccgtacacggcgggggctcagcagtacgttagtctggacctgcggccgctggaggtgcggcgggggctgcgcgcccaggcctgcgccttctggaaccgcttcctccccaaattgctcagcgccaccgacacgctcgacgaggcggagcgccagtggaaggccgagttccaccgctggagctgytacatggtgcactggaagaaccagttcgaccactacagcaagcaggatcgctgctcagacctgtgaccccggcgggacccccatgtcctccgctccgcccggccccctagctgtatatactatttatttcagggctgggctataacacagacgagccccagactctgcccatccccaccccaccccgacgtcccccggggctcccggtcctctggcatgtcttcaggctgagctcctccccgcgtgccttcgccctctggctgcaaataaactgttacaggcc

SEQ ID NO: 6 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSCYMVHWKNQFDHYSKQDRCSDL

SEQ ID NO: 7 - DNA, 1815 bp, Homo sapiens
  CDS (7)..(1812)
gccaccatggatagcaaagtcacaatcatatgcatcagatttctcttttggtttcttttgctctgcatgcttattgggaagtcacatactgaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggttcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaactgytacatgatggactggaaaaatcaatttaacgattacactagcaagaaagaaagttgtgtgggtctctaa

SEQ ID NO: 8 - protein, 602 aa, Homo sapiens
MDSKVTIICIRFLFWFLLLCMLIGKSHTEDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNCYMMDWKNQFNDYTSKKESCVGL

SEQ ID NO: 9 - DNA, 2066 bp, Rattus rattus
  CDS (1)..(1842)
atgaggcctccctggtatcccctgcatacaccctccctggcttctccactcctcttcctcctcctctccctcctgggaggaggggcaagggctgagggccgggaagaccctcagctgctggtgagggttcgagggggccagctgaggggcatccgcctgaaggcccctggaggcccagtctcagcttttctgggcatcccctttgcagagccacctgtgggctcacgtagatttatgccaccagagcccaagcgcccctggtcaggaatattggatgctaccaccttccaaaatgtctgctaccaatacgtggacaccctgtaccctgggtttgagggtaccgagatgtggaaccccaatcgagagctgagtgaagactgcctttatcttaatgtgtggacaccataccccaggcctacttctcccacacctgtcctcatctggatctatgggggtggtttctacagtggagcatcctccttggacgtgtatgacggccgtttcctggcccaggttgagggaaccgtgttggtatctatgaactaccgagtgggaacctttggcttcttggctctaccaggaagcagagaagcccctggcaatgtaggcctgctggatcaacggcttgccttgcaatgggtacaagaaaatatcgcagcctttgggggagacccaatgtcagtgactctgtttggggagagtgcaggtgcagcctcagtgggcatgcacattctgtccctgcccagcaggagcctcttccacagggctgtcctgcagagtggcacacccaatgggccctgggccactgtgagtgcgggagaggccaggcgcagggccacactgctggcccgccttgtgggctgtcccccaggtggcgctggtggcaatgacaccgagctgatatcctgcttgaggacaaggcccgctcaggacctggtggaccacgagtggcatgtgctgcctcaagaaagtatcttccggttttccttcgtgcctgtggtggacggggatttcctcagtgacacgccggacgctctcatcaatactggagattttcaagacctgcaggtgctggtgggtgtggtgaaggacgagggctcctactttctggtttacggggtcccaggcttcagcaaagacaatgaatctctcatcagccgggcccagttcctggctggggtgcggatcggtgtaccccaagcgagtgacctggcggccgaggctgtggtcctgcattatacagactggctgcaccctgaggaccctgcccacctgagagatgccatgagtgcggtggtaggcgaccacaacgttgtgtgccctgtggcccagctggctgggcgactggctgcccaaggggctcgggtctatgcctacatctttgaacaccgtgcctccacattgacttggcccctctggatgggggtgccccatggctatgaaatcgagttcatctttgggctccccctggatccctcactgaactacaccgtggaggagagaatctttgctcagcgacttatgcaatactggaccaattttgcccgcacaggggaccccaatgaccctcgagactctaagtctccacggtggccaccgtacaccactgccgcgcagcaatacgtgagcctgaacctgaagcctttggaggtgcggcggggactgcgcgcccagacctgcgccttctggaatcgttttctccccaaattgctcagcgccacagacacgctggacgaggcggagcgccagtggaaggccgagttccaccgctggagctgytacatggtgcactggaagaaccagttcgaccactatagcaagcaggaacgctgctcagacctgtgaccccttggggacccaggtcctgccgtcctgcccgagcccctgattgtatatacactatttatttaagggctgggatataatacaaccgagcccccaggccctgtccacccctccccgacttcctcccactaggggatcctcatcttctgcatgttttaaactgagctcccctccccgcggtgccttgccccctctgggccgccaataaactgttacagctc

SEQ ID NO: 10 - protein, 614 aa, Rattus rattus
MRPPWYPLHTPSLASPLLFLLLSLLGGGARAEGREDPQLLVRVRGGQLRGIRLKAPGGPVSAFLGIPFAEPPVGSRRFMPPEPKRPWSGILDATTFQNVCYQYVDTLYPGFEGTEMWNPNRELSEDCLYLNVWTPYPRPTSPTPVLIWIYGGGFYSGASSLDVYDGRFLAQVEGTVLVSMNYRVGTFGFLALPGSREAPGNVGLLDQRLALQWVQENIAAFGGDPMSVTLFGESAGAASVGMHILSLPSRSLFHRAVLQSGTPNGPWATVSAGEARRRATLLARLVGCPPGGAGGNDTELISCLRTRPAQDLVDHEWHVLPQESIFRFSFVPVVDGDFLSDTPDALINTGDFQDLQVLVGVVKDEGSYFLVYGVPGFSKDNESLISRAQFLAGVRIGVPQASDLAAEAVVLHYTDWLHPEDPAHLRDAMSAVVGDHNVVCPVAQLAGRLAAQGARVYAYIFEHRASTLTWPLWMGVPHGYEIEFIFGLPLDPSLNYTVEERIFAQRLMQYWTNFARTGDPNDPRDSKSPRWPPYTTAAQQYVSLNLKPLEVRRGLRAQTCAFWNRFLPKLLSATDTLDEAERQWKAEFHRWSCYMVHWKNQFDHYSKQERCSDL

SEQ ID NO: 11 - DNA, 2357 bp, Torpedo marmorata
  CDS (178)..(1974)
gaattctctgattcatccagggcttctggctggggaacacccggtatgttcgcgtgggcacacactcatccacgcaggtcttgatgaagtcggtgacacctgttgcatattcattcaagtctatgggcagcagggatctttgccttccttgacaagctggagagttgcaaagcagacatgagagaaatgaatctgctggtcacctcttcgctgggcgtgcttctgcacttggtcgtcctgtgccaggcggacgatgactctgagctcctggtcaacaccaagtcgggaaaagtcatgcgaacaagaatccctgtcctctccagccacatcagcgctttcctggggattccctttgccgagcctccagttgggaacatgagattcaggagacctgagcccaagaaaccgtggtcgggagtctggaatgcttccacctatcccaacaactgccagcagtacgttgacgagcagttccctggatttccaggttcggagatgtggaatccgaacagagagatgagtgaggactgtttgtacctcaacatttgggtgccttctccgaggccgaagagtgcaaccgtcatgttgtggatctacggaggcggtttctacagcgggtcctcgacgttggacgtctacaatgggaaataccttgcctacaccgaggaggtggtgctggtctctctgagctaccgggtgggcgcttttggctttctcgccctccacggcagtcaggaggcaccaggaaacatgggcctcctggaccagaggatggcgctgcagtgggtgcacgacaacatccagttcttcggcggggaccccaagacggtgaccctcttcggagagagtgccggccgcgcctctgtcggcatgcacattctctccccggggagccgagacctcttccgccgggccatccttcagagcggctcgcccaattgcccgtgggcatctgtctctgttgctgaaggccgcaggagggcggtcgagctgcgaagaaacctcaactgcaacctcaacagcgacgaagacctcatccaatgtcttagggagaagaagcctcaggagttgattgacgtggagtggaatgtccttccctttgacagtatcttcaggttttccttcgttcccgtcatcgatggggaattcttcccaacctccctggaatctatgttgaacgctggcaacttcaagaagactcagatcttactgggagtcaacaaggacgagggctcgtttttcctcttgtacggagcgccgggtttcagcaaggactctgaaagcaaaatctctcgggaagacttcatgtcaggggtcaagctaagcgttccccacgccaatgacttagggttggacgctgtcacgctacagtacacagactggatggatgacaacaatggaataaagaacagagatggattggacgacatcgtaggggaccacaacgtcatatgccctttgatgcactttgttaacaagtacaccaagtttggtaatggcacctacctgtacttcttcaaccaccgagcctcaaacctggtgtggccggagtggatgggcgtcatccacggctatgagattgagttcgtcttcgggctgcctctggtgaaggagctgaactacacagcggaggaggaagcgctgagccggaggataatgcattactgggcgacattcgcaaagactggaaacccaaacgaaccccactcacaggagagcaaatggcctctcttcactaccaaggagcagaaatttattgacctcaacacagagcccataaaagtccaccagcgactccgagttcagatgtgcgtattctggaaccagttcctccccaagctcctcaacgccacagagaccattgacgaggcagaacgccagtggaagacggagtttcatcggtggagttgytacatgatgcactggaagaaccaatttgaccagtacagcagacacgagaactgtgctgagctgtgagctctgcactgcaggtcgcctggtgaggcagagagcagagtccgatattgaccagacacccagtctagttcctggagaccctgcctggccctctgagccaccccgccaaacctcgccttcgagactccctccctcccatccctccgctaaccccaggctgccgcaccttgttctctcgcatcatcactcgtgaatctgcggccaccattctttcggccacccactctcgcatcagcccaattctctccttcctctcccccccctattgcccactctccacctgccgcactctctctcccaatacccgtcagcaaccattccggtccccttgatctctctcctgtcacctattgacgttctacccaaccccccctactcgg

SEQ ID NO: 12 - protein, 599 aa, Torpedo marmorata
MREMNLLVTSSLGVLLHLVVLCQADDDSELLVNTKSGKVMRTRIPVLSSHISAFLGIPFAEPPVGNMRFRRPEPKKPWSGVWNASTYPNNCQQYVDEQFPGFPGSEMWNPNREMSEDCLYLNIWVPSPRPKSATVMLWIYGGGFYSGSSTLDVYNGKYLAYTEEVVLVSLSYRVGAFGFLALHGSQEAPGNMGLLDQRMALQWVHDNIQFFGGDPKTVTLFGESAGRASVGMHILSPGSRDLFRRAILQSGSPNCPWASVSVAEGRRRAVELRRNLNCNLNSDEDLIQCLREKKPQELIDVEWNVLPFDSIFRFSFVPVIDGEFFPTSLESMLNAGNFKKTQILLGVNKDEGSFFLLYGAPGFSKDSESKISREDFMSGVKLSVPHANDLGLDAVTLQYTDWMDDNNGIKNRDGLDDIVGDHNVICPLMHFVNKYTKFGNGTYLYFFNHRASNLVWPEWMGVIHGYEIEFVFGLPLVKELNYTAEEEALSRRIMHYWATFAKTGNPNEPHSQESKWPLFTTKEQKFIDLNTEPIKVHQRLRVQMCVFWNQFLPKLLNATETIDEAERQWKTEFHRWSCYMMHWKNQFDQYSRHENCAEL

SEQ ID NO: 13 - DNA, 2089 bp, Mus musculus
  CDS (1)..(1842)
atgaggcctccctggtatcccctgcatacaccttccctggcttttccactcctcttcctcctcctctccctcctgggaggaggggcaagggctgagggccgggaagacccgcagctgctggtgagggttcgagggggccagctgaggggcatccgcctgaaggcccctggaggcccagtctcagcttttctgggcatcccctttgcagagccacctgtgggctcacgtagatttatgccaccagagcccaagcggccctggtcaggagtgttggatgctaccaccttccaaaatgtctgctaccagtacgtggacaccctgtaccctgggtttgagggtactgagatgtggaaccccaaccgagagttgagtgaagactgcctgtatcttaatgtgtggacaccataccccagacctgcttctcccacacctgtcctcatctggatctatgggggtggtttctacagcggagcggcctccttggatgtgtatgacggccgtttcctggcccaggttgagggagctgtgttggtatctatgaactaccgagtgggaacctttggcttcttggccctaccaggaagcagagaagcccctggcaatgtaggtctgctggatcaacggcttgccttgcaatgggtgcaagaaaatattgcagcctttgggggcgacccgatgtcagtgactctgtttggggagagtgcgggtgcagcctccgtgggcatgcacatactgtccctgcccagcaggagcctcttccacagggctgtcctccagagtggcacacccaatgggccctgggccactgtgagtgctggagaggccaggcgcagggccacactgctggcccgccttgtgggctgtcccccaggtggcgctggtggcaatgacaccgagctgatagcctgcttgaggacaaggcccgctcaggacctggtggaccacgagtggcacgtcctgcctcaagaaagtatcttccgattttccttcgtgcctgtggtagacggggacttcctcagtgacacaccggaggctctcatcaatactggagattttcaagacctgcaggtgctggtgggtgtggtgaaggacgagggctcctactttctggtttacggggtcccaggcttcagcaaagacaatgaatctctcatcagccgggcccagttcctggctggggtgcggatcggtgtaccccaagcaagtgacctggcggccgaggctgtggtcctgcattacacagactggttgcaccctgaggaccctactcacctgagagatgccatgagtgcagtggtaggcgaccacaacgttgtgtgccctgtggcccagctggctgggcgactggctgcccaaggggcccgggtctatgcctacatctttgaacaccgtgcctccacactgacttggcccctctggatgggggtgccccatggctatgaaatcgagttcatctttgggctccccctggatccctcgctgaactacaccacggaggagaggatctttgctcagcgacttatgaaatactggaccaattttgcccgcacaggggaccccaatgaccctcgagactccaaatctccacagtggccaccgtacaccactgccgcgcagcaatatgtgagcctgaacctgaagcccttagaggtgcggcggggactgcgcgcccagacctgcgccttctggaatcgctttctccccaaattgctcagcgccaccgatactctggacgaggcggagcgccagtggaaggccgagttccaccgctggagctgytacatggtgcactggaagaaccagttcgaccactatagcaagcaggagcgctgctcagacctgtgaccccttggggaccccaggtcctgccgccctgcccgagcccctagctgtatatacactatttatttaagggctgggatataatacgaccgagcccccaggccctgtccactcctccccgacttcctcccactaggggctccccatcttctgcatgtcttgggctaagctcccctccccgcggtgccttcgcccctctgggccgccaataaactgttacagccaccaaaaaaaaaaaaaaaaaaaa

SEQ ID NO: 14 - protein, 614 aa, Mus musculus
MRPPWYPLHTPSLAFPLLFLLLSLLGGGARAEGREDPQLLVRVRGGQLRGIRLKAPGGPVSAFLGIPFAEPPVGSRRFMPPEPKRPWSGVLDATTFQNVCYQYVDTLYPGFEGTEMWNPNRELSEDCLYLNVWTPYPRPASPTPVLIWIYGGGFYSGAASLDVYDGRFLAQVEGAVLVSMNYRVGTFGFLALPGSREAPGNVGLLDQRLALQWVQENIAAFGGDPMSVTLFGESAGAASVGMHILSLPSRSLFHRAVLQSGTPNGPWATVSAGEARRRATLLARLVGCPPGGAGGNDTELIACLRTRPAQDLVDHEWHVLPQESIFRFSFVPVVDGDFLSDTPEALINTGDFQDLQVLVGVVKDEGSYFLVYGVPGFSKDNESLISRAQFLAGVRIGVPQASDLAAEAVVLHYTDWLHPEDPTHLRDAMSAVVGDHNVVCPVAQLAGRLAAQGARVYAYIFEHRASTLTWPLWMGVPHGYEIEFIFGLPLDPSLNYTTEERIFAQRLMKYWTNFARTGDPNDPRDSKSPQWPPYTTAAQQYVSLNLKPLEVRRGLRAQTCAFWNRFLPKLLSATDTLDEAERQWKAEFHRWSCYMVHWKNQFDHYSKQERCSDL

SEQ ID NO: 15 - DNA, 126 bp, Homo sapiens
  CDS (1)..(123)
ggaaatattgatgaagcagaatgggagtggaaagcaggattccatcgctggaactgytacatgatggactggaaaaatcaatttaacgattacactagcaagaaagaaagttgtgtgggtctctaa

SEQ ID NO: 16 - protein, 41 aa, Homo sapiens
GNIDEAEWEWKAGFHRWNCYMMDWKNQFNDYTSKKESCVGL

SEQ ID NO: 17 - DNA, 123 bp, Rattus rattus
  CDS (1)..(120)
gacacgctggacgaggcggagcgccagtggaaggccgagttccaccgctggagctgytacatggtgcactggaagaaccagttcgaccactatagcaagcaggaacgctgctcagacctgtga

SEQ ID NO: 18 - protein, 40 aa, Rattus rattus
DTLDEAERQWKAEFHRWSCYMVHWKNQFDHYSKQERCSDL

SEQ ID NO: 19 - DNA, 123 bp, Torpedo marmorata
  CDS (1)..(120)
gagaccattgacgaggcagaacgccagtggaagacggagtttcatcggtggagttgytacatgatgcactggaagaaccaatttgaccagtacagcagacacgagaactgtgctgagctgtga

SEQ ID NO: 20 - protein, 40 aa, Torpedo marmorata
ETIDEAERQWKTEFHRWSCYMMHWKNQFDQYSRHENCAEL

SEQ ID NO: 21 - DNA, 123 bp, Mus musculus
  CDS (1)..(120)
gatactctggacgaggcggagcgccagtggaaggccgagttccaccgctggagctgytacatggtgcactggaagaaccagttcgaccactatagcaagcaggagcgctgctcagacctgtga

SEQ ID NO: 22 - protein, 40 aa, Mus musculus; identical to SEQ ID NO: 18

SEQ ID NO: 23 - protein, 40 aa, Homo sapiens
DTLDEAENQWKCEFHRWSSYMVHWKNQFDHYSKQDRCSDL

SEQ ID NO: 24 - protein, 40 aa, Homo sapiens
DTLDEAENQWKACFHRWSSYMVHWKNQFDHYSKQDRCSDL

SEQ ID NO: 25 - protein, 40 aa, Homo sapiens
DTLDEAENQWKAECHRWSSYMVHWKNQFDHYSKQDRCSDL

SEQ ID NO: 26 - protein, 40 aa, Homo sapiens
DTLDEAENQWKAEFCRWSSYMVHWKNQFDHYSKQDRCSDL

SEQ ID NO: 27 - protein, 40 aa, Homo sapiens
DTLDEAENQWKAEFHCWSSYMVHWKNQFDHYSKQDRCSDL

SEQ ID NO: 28 - protein, 40 aa, Homo sapiens
DTLDEAENQWKAEFHRCSSYMVHWKNQFDHYSKQDRCSDL

SEQ ID NO: 29 - protein, 40 aa, Homo sapiens
DTLDEAENQWKAEFHRWCSYMVHWKNQFDHYSKQDRCSDL

SEQ ID NO: 30 - protein, 8 aa, Artificial Sequence
  Description of Artificial Sequence Synthetic peptide
DYKDDDDK